(19) United States
(12) Patent Application Publication
(10) Pub. No.: US 2016/0381733 A1
(43) Pub. Date: Dec. 29, 2016

(54) COMBINED LIGHTING AND HEATING DEVICE

(71) Applicants: Myoung Hee PARK, Siheung-si Gyeonggi-do (KR); Byung Hack LEE, Siheung-si Gyeonggi-do (KR); Kwang Sung LEE, Yeonsu-gu Incheon (KR); SHINHAN ELECTRIC, Siheung-si Gyeonggi-do (KR); K2LENG CO., LTD., Siheung-si Gyeonggi-do (KR)

(72) Inventors: Byung Hack LEE, Siheung-si (KR); Kwang Sung LEE, Incheon (KR)

(21) Appl. No.: 15/117,468

(22) PCT Filed: Jan. 20, 2015
(86) PCT No.: PCT/KR2015/000611

§ 371 Date: Aug. 9, 2016

(30) Foreign Application Priority Data
Feb. 10, 2014 (KR) 10-2014-0015072

(51) Int. Cl. H05B 3/00; F21V 29/90; A61N 5/06; H05B 37/02; A61L 9/18; F21V 33/00; F21V 25/12

(52) U.S. Cl. CPC H05B 3/0085; H05B 3/008; F21V 33/0092; F21V 29/90; F21V 25/12; H05B 37/0272; A61L 9/18; A61N 5/0613; F21Y 2115/10

(57) ABSTRACT

The present invention relates to a combined lighting and heating device. To this end, the present invention provides a combined lighting and heating device, in which a heating means (20) and an LED lamp (30) are included in a frame (10) of a heating lighting device (1) attached to a ceiling or a wall surface so as to neutralize contaminants by far-infrared radiation energy and to raise the uniform indoor temperature by a heating function of a lighting device. The present invention having the above configuration neutralizes contaminants by far-infrared radiation energy and raises the uniform indoor temperature by the heating function of the lighting device, thereby significantly improving the quality and reliability of a product and satisfying various demands (needs) of consumers who use the product so as to create a good product image.

### TECHNICAL FIELD

The present invention relates to a combined lighting and heating device, and in particular to a combined lighting and heating device wherein contaminants can be neutralized using a far infrared ray radiant energy, and a uniform indoor temperature can be raised using a heating function of a lighting equipment itself, whereupon a product quality and reliability can be significantly enhanced, thus satisfying various needs of customers, who are actual users, and providing a good impression.

### BACKGROUND ART

As building materials are being developed better and better in a modern society, a lot of buildings can be built in a more convenient way, and in particular, newly designed buildings are being constructed endlessly so far with the aid of standardized materials and conveniences.

The conventional technology has a problem in the way that though such materials are convenient to use, there are too many synthesized products. For this reason a new skin disease which has not been yet reported in the field of an old building, is occurring, and the indoor air contamination in a closed space is becoming worse. This situation may cause any exposure to a health risk, for example, atopy and a house dust mite, due to a carcinogen, and people are suffering from an unknown disease which has not yet been found in an actual life.

In a conventional building heating system, heating has been carried out by combusting fossil fuel, for example, petroleum, gas, etc., but the gas generating during a fuel combustion causes an air contamination, and an overheating is inevitable if it needs to raise an indoor temperature, for which a lot of energy loss may occur.

In order to resolve the aforementioned problems, a technology described in the following prior art document is filed, but the problems still remain unresolved.

### PRIOR ART TECHNOLOGY DOCUMENT

### Patent Document

(Patent document 1) The Korean patent laid-open No. 2010-0120499 (Nov. 16, 2010) (Title: a far infrared ray heating device and a far infrared ray heating and lighting device including the same) is disclosed.

### DISCLOSURE OF INVENTION

### Technical Problem

Accordingly, the present invention is made in an effort to resolve the aforementioned problems. It is a first object of the present invention to provide a heating and lighting device which may include a frame, a heating means, a

LED lamp, a temperature control unit, and a remote controller. A second object of the present invention is to deodorize and neutralize a volatile formaldehyde which is a cause of sick house syndrome in such a way to radiate and heat an indoor temperature with the aid of a far infrared ray radiant energy, thus maximizing and evenly raising efficiency at 40˜60°. A third object of the present invention is to activate the metabolism of a human body by penetrating a far infrared ray radiant energy into a human body and an organism and making an environment-friendly indoor environment providing a deodorization function and a sterilization function via a fresh indoor environment. A fourth object of the present invention is to develop a LED lighting device and a far infrared ray heating method into a module type and use them selectively if necessary, and significantly reduce the use of energy by recognizing in real time an indoor air contamination and the amount of electricity use. A fifth object of the present invention is to save the costs for an indoor lighting and a heating facility in such a way to combine a far infrared ray heating device which is able to radiate a far infrared ray, and a lighting device, and is to provide an effect wherein any maintenance of a product is not necessary. A sixth object of the present invention is to increase a market share ratio in the whole markets with the aid of strengthened compatibility in the domestic and foreign markets based on the possession of a product asset having a differentiated design. A seventh object of the present invention is to provide a good impression by satisfying various needs from a customer, who is an actual use of the product, since the quality and reliability of the product can be greatly enhanced based on the aforementioned features.

### Technical Solution

To achieve the above objects, there is provide a combined lighting and heating device wherein a heating means and a LED lamp are provided at a frame of a heating and lighting device which is attached on a ceiling or a wall surface in order to neutralize any contaminants using a far infrared ray radiant energy and raise a uniform indoor temperature with the aid of a heating function of a lighting equipment itself.

### Advantageous Effects of the Invention

As mentioned earlier, the present invention provided a heating and lighting device which may include a frame, a heating means, a LED lamp, a temperature control unit, and a remote controller.

The present invention having the above-described technical configuration is able to deodorize and neutralize a volatile formaldehyde which is a cause of sick house syndrome in such a way to radiate and heat an indoor temperature with the aid of a far infrared ray radiant energy, thus maximizing and evenly raising efficiency at 40˜60°.

In particular, the present invention is able to activate the metabolism of a human body by penetrating a far infrared ray radiant energy into a human body and an organism and making an environment-friendly indoor environment providing a deodorization function and a sterilization function via a fresh indoor environment.

Moreover, the present invention is able to develop a LED lighting device and a far infrared ray heating method into a module type and use them selectively if necessary, and significantly reduce the use of energy by recognizing in real time an indoor air contamination and the amount of electricity use.

In addition, the present invention is able to save the costs for an indoor lighting and a heating facility in such a way to combine a far infrared ray heating device which is able to radiate a far infrared ray, and a lighting device, and is to provide an effect wherein any maintenance of a product is not necessary.

Furthermore, the present invention is able to increase a market share ratio in the whole markets with the aid of strengthened compatibility in the domestic and foreign markets based on the possession of a product asset having a differentiated design.

The present invention is a very useful invention since it is able to provide a good impression by satisfying various needs from a customer, who is an actual use of the product, since the quality and reliability of the product can be greatly enhanced based on the aforementioned features.

The preferred embodiments of the present invention to achieve the above-listed effects will be described with reference to the accompanying drawings.

### BRIEF DESCRIPTION OF DRAWINGS

FIG. 1 is a cross sectional view illustrating a major component of a lighting and heating device according to the present invention.

FIG. 2 is a block diagram illustrating a temperature controller according to the present invention.

FIG. 3 is a view illustrating a use state of a heating and lighting device and a temperature controller according to the present invention.

### MODES FOR CARRYING OUT THE INVENTION

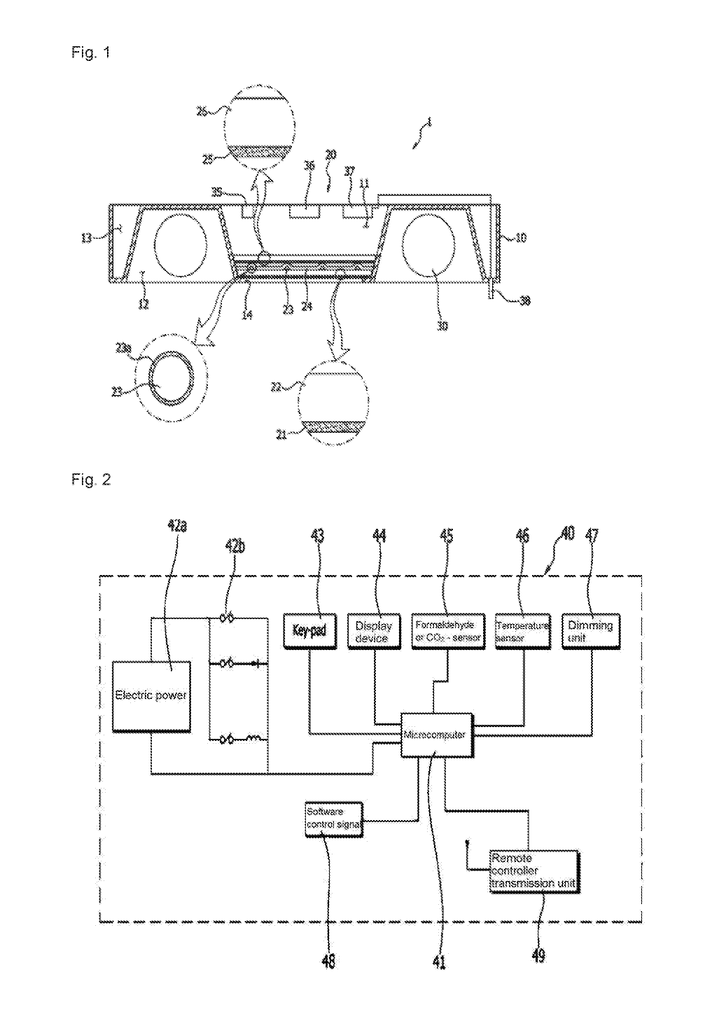

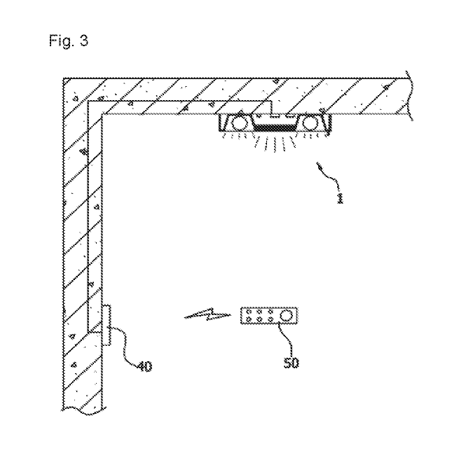

As illustrated in FIG. 1 or FIG. 3, the combined lighting and heating device according to the present invention will be described with reference to the accompanying drawings.

Throughout the description of the present invention, if it is judged that the detailed descriptions on the related known function or configuration may make unclear the subject matter of the present invention, the aforementioned descriptions will be omitted.

The terms used later are the terms which are defined given the functions disclosed in the present invention, and such terms may change based on a manufacturer's intension or custom, so the definitions thereon should be set based on the whole contexts of the present specification.

As illustrated in FIG. 1, the present invention provides a combined lighting and heating device wherein a heating means 20 and a LED lamp 30 are provided at a frame 10 of a heating and lighting device 1 which is attached on a ceiling or a wall surface in order to neutralize any contaminants using a far infrared ray radiant energy and raise a uniform indoor temperature using a heating function of a lighting equipment itself.

The frame 10 according to the present invention may include a first space part 11 configured to accommodate the heating means 20 therein, a second space part 12 configured to accommodate the LED lamp 30 therein, a third space part 13 to cool the heat of the LED lamp, and a hole 14 configured to pass a far infrared ray radiant energy and a heat of the heating means 20 and supply them into an indoor space.

The heating means 20 according to the present invention may include a first plate 22 which is disposed at the top of the hole 14 in order for the far infrared ray radiant energy and the heat to pass downward, wherein the lower side of the first plate 22 is coated with a far infrared ray radiation coating part 21.

Moreover, a heating hot wire 23 is provided at the top of the first plate 22 and is made of a Teflon material which is able to radiate heat.

It is preferred that the heating hot wire 23 includes a silicon 23a which is able to withstand a temperature of up to 200˜500° C. and is able to easily attach to a taping member.

Moreover, a taping member 24 is provided at the top of the heating hot wire 23 and is employed to fix a heating hot wire.

Furthermore, a second plate 26 is provided at the top of the taping member 24 and is employed to shield the heat which radiates upward, wherein a heat-shielding coating part 25 is coated on the lower side of the second plate 26.

In addition, the far infrared ray radiation coating part 21 according to the present invention is coated with a mixture substance mixed with a liquid fireproof paint, carbons and a radiation substance.

The mixture substance may contain a quantum energy paint which allows to increase a human body immunity by generating a biological energy, which contains SiO₂ of 50-65% by weight, Al₂O₃ of 25-31% by weight, Fe₂O₃ of 2-5% by weight, CaO of 1-2% by weight, MgO of 1-2% by weight, K₂O of 3-5% by weight, and Na₂O of 3-5% by weight.

In particular, if the content of SiO₂ is smaller than 50% by weight, the far infrared ray effects and waveforms may decrease, and if it is over 65% by weight, it does not melt with other components. For this reason, it is preferred that the content of SiO₂ is 50-65% by weight.

Moreover, if the content of Al₂O₃ is smaller than 25% by weight, the catalyst reaction may become bad, and if it is over 31% by weight, it can become easily hardened. For this reason, it is preferred that the content of Al₂O₃ is 25-31% by weight.

Furthermore, if the content of Fe₂O₃ is smaller than 2% by weight, a heat radiation power may become bad, and if it is over 5% by weight, it is not solidified well with other components. For this reason, it is preferred that the content of Fe₂O₃ is 2-5% by weight.

In addition, if the content of CaO is smaller than 1% by weight, a mixing power with other components may become bad, and if it is over 2% by weight, there may be a danger in terms of suction since alkaline is strong. For this reason, it is preferred that the content of CaO is 1-2% by weight.

Moreover, if the content of MgO is smaller than 1% by weight, a suction power may become bad, and if it is over 2% by weight, a mixing power with other components may become bad. For this reason, it is preferred that the content of MgO is 1-2% by weight.

Furthermore, if the content of K₂O is smaller than 3% by weight, an absorption property may become bad, and if it is over 5% by weight, a reaction performance may become bad. For this reason, it is preferred that the content of K₂O is 3-5% by weight.

Finally, if the content of Na₂O is smaller than 3% by weight, it may be mixed well with other components, but may not be melted, and if it is over 5% by weight, it may not be mixed with other components. For this reason, the content of Na₂O is 3-5% by weight.

Moreover, the heat-shielding coating part 25 according to the present invention may be coated with a Teflon material for the sake of a heat-resisting property and an insulation safety, while interrupting the transfer of heat in the upward direction.

As illustrated in FIG. 3, the heating and lighting device 1 according to the present invention is connected to a temperature controller 40 configured to maintain temperature constant, and the temperature controller 40 is connected to a remote controller 50 wherein a user is able to control in person using switches or is able to control remotely.

Moreover, as illustrated in FIG. 2, the temperature controller 40 according to the present invention is equipped with a microcomputer 41 which is able to control the whole parts of the temperature controller. The microcomputer 41 may be connected with an electric power 42a and a switch 42b, a key pad 43 which is an input handling device, a display device 44 which may be formed of a display able to visually display data or a touch panel that a user is able to input in person while watching a screen, a sensor 45 configured to detect formaldehyde or carbon dioxide, a temperature sensor 46, a dimming unit 47 to control the environment of a lamp, a software control signal 48, and a remote controller transmission unit 49.

Meanwhile, the frame 10 according to the present invention may be equipped with an electric power terminal 35, and a power supply device (SMPS) 36 and may be further equipped with an anion module 37 which is able to generate anion of about 20,000/1 cc of air, thus carrying out sterilization and deodorization operations while purifying indoor air. The anion module 37 is able to carry out a corona discharge by limiting ozone below 0.05 ppm. It is preferred that the anion module is connected with an anion sensor 38.

The present invention may be change into various forms when actually employing the above-described components and may use various types.

It should be understood that the present invention is not limited to the specific forms which have been described throughout the descriptions, but the present invention should be understood as including all modifications, equivalents and substitutes which match with the spirit of the present invention and are in the scope of the present invention.

The operations and effects of the combined lighting and heating device according to the present invention will be described.

First, the present invention makes it possible to neutralize any contaminants using a far infrared ray radiant energy and raise a uniform indoor temperature through the heating function of a lighting equipment itself.

To this end, when the user drives the heating and lighting device 1 by operating the remote controller 50 or directly the temperature controller 40, the heating and lighting device 1 generates a far infrared ray radiant energy, thus neutralizing any contaminants, and the uniform indoor temperature can be raised by the heating function of the lighting equipment itself. In this invention, the heating and lighting device 1 may be used attached to a ceiling as illustrated in FIG. 3 or a wall surface (not illustrated in the drawing), by means of which it is possible to provide the same effects as the aforementioned effects.

As illustrated in FIG. 2, the temperature controller 40 according to the present invention is able to control and drive the heating and lighting device 1 with the aid of the electric power 42a and the switch 42b. The microcomputer 41 may be connected with a key pad 43 which is an input handling device, a display device 44 which may be formed of a display able to visually display data or a touch panel that a user is able to input in person while watching a screen, a sensor 45 configured to detect formaldehyde or carbon dioxide, a temperature sensor 46, a dimming unit 47 to control the environment of a lamp, a software control signal 48, and a remote controller transmission unit 49 and may drive the above listed components.

Meanwhile, the heating and lighting device 1 according to the present invention is configured to neutralize any contaminants and raise a uniform indoor temperature as described above. This operation will be described in more details.

First, as an electric power is supplied, the heating hot wire 23 is heated. The first plate 22 is heated by a high temperature heat of about 70˜90° C., and the first plate 22 may heat the far infrared ray radiation coating part 21. Since the far infrared ray radiation coating part 21 is coated with a mixture mixed with a liquefied fireproof paint, carbons and a radiation substance, thus generating heat. A far infrared ray may generate due the generated heat, and may be transferred into the indoor space.

Moreover, the heating hot wire 23 according to the present invention is installed fixed by the taping member 24, so it should not be movable or disengaged.

Furthermore, the second plate 26 and the heat-shielding coating part 25 according to the present invention are provided to allow the heat generating by the heating hot wire 23 to move only in the downward direction, not in the upward direction, thus preventing any loss of heat.

More specifically, the heat-shielding coating part 25 is coated with a Teflon material, thus preventing the heat from being transferred in the upward direction, while providing a heat-resisting property and an insulation safety.

Moreover, the frame 10 according to the present invention is formed of the first space part 11 to accommodate the heating means 20, the second space part 12 to accommodate the LED lamp, the third space part 13 to cool the heat of the LED lamp, and the hole 14 which is configured to pass a far infrared ray radiant energy and heat of the heating means 20 and supply it to the indoor space. The indoor air can be heated by the heat generating from the LED lamp 30.

### DESCRIPTIONS ON THE REFERENCE NUMBERS OF MAJOR COMPONENTS IN THE DRAWINGS

Legend of Reference Number

| | |
|---|---|
| 1: Heating and lighting device | 10: Frame |
| 20: Heating means | 30: LED lamp |
| 40: Temperature controller | 50: Remote controller |

### INDUSTRIAL APPLICABILITY

The technical concept of the combined lighting and heating device according to the present invention is able to repeatedly carry out the same results, and the present invention may accelerate a technical development through the implementation of the present invention, so the present invention may contribute to the industrial development, while providing enough protective values.

What is claimed is:

1. A combined lighting and heating device, comprising:
a heating means 20, a LED (Light Emitting Diode) lamp 30, and a temperature controller 40 which is provided to maintain temperature constant, which are included in a frame 10 of a heating and lighting device 1 attached on a ceiling or a wall surface so as to neutralize any contaminants using a far infrared ray energy and raise a uniform indoor temperature via a heating function of a lighting equipment, wherein the frame 10 includes a first space part 11 for accommodating the heating means 20, a second space part 12 for accommodating the LED lamp 30, a third space part 13 for cooling the heat from the LED lamp, and a hole 14 for passing the infrared ray radiant energy and heat of the heating means 20, and the heating means 20 includes a first plate 22 which is disposed at the top of the hole 14 so as to pass the far infrared ray radiant energy and the heat in the downward direction, wherein the lower side of the first plate 22 is coated with a far infrared ray radiation coating part 21 is coated, a heating hot wire 23 which is disposed at the top of the first plate and is made of a Teflon material which is able to generate heat, a taping member 24 which is disposed at the top of the heating hot wire and is able to fix the heating hot wire, and a second plate 26 which is disposed at the top of the taping member and is configured to interrupt the radiation of the heat in the upward direction, wherein the lower side of the second plate 26 is coated with a heat-shielding coating part 25, and the far infrared ray radiation coating part 21 is coated with a mixture substance mixed with a liquefied fireproof paint, carbons, and a radiation substance, wherein the heating hot wire 23 contains a silicon 23a which is able to withstand a temperature of 200˜500° C. and is fixedly attached to the taping member.

2. The device of claim 1, wherein the mixture substance contains a quantum energy paint which allows to increase a human body immunity by generating a biological energy, which contains SiO₂ of 50-65% by weight, Al₂O₃ of 25-31% by weight, Fe₂O₃ of 2-5% by weight, CaO of 1-2% by weight, MgO of 1-2% by weight, K₂O of 3-5% by weight, and Na₂O of 3-5% by weight.

3. The device of claim 1, wherein the heat-shielding coating part 25 is coated with a Teflon material so as to prevent heat from transferring in the upward direction, while providing a heat-resisting property and an insulation safety.

4. The device of claim 1, wherein the temperature controller 40 is equipped with a microcomputer 41 which is able to control the whole parts of the temperature controller, and the microcomputer 41 is connected with an electric power 42a and a switch 42b, a key pad 43 which is an input handling device, a display device 44 which is formed of a display able to visually display data or a touch panel that a user is able to input in person while watching a screen, a sensor 45 configured to detect formaldehyde or carbon dioxide, a temperature sensor 46, a dimming unit 47 to control the environment of a lamp, a software control signal 48, and a remote controller transmission unit 49.

5. The device of claim 1, wherein the frame 10 is equipped with an electric power terminal 35, and a power supply device (SMPS) 36 and is further equipped with an anion module 37 which is able to generate anion of about 20,000/1 cc of air, thus carrying out sterilization and deodorization operations while purifying indoor air, and the anion module 37 is able to carry out a corona discharge by limiting ozone below 0.05 ppm, and the anion module is connected with an anion sensor 38.
